(19) United States
(12) Patent Application Publication
(10) Pub. No.: US 2009/0105614 A1
(43) Pub. Date: Apr. 23, 2009

(54) ANALYTE COLLECTION CHIP

(75) Inventors: Shun Momose, Kyoto (JP); Takaaki Shimasaki, Kyoto (JP)

Correspondence Address: GLOBAL IP COUNSELORS, LLP, 1233 20TH STREET, NW, SUITE 700, WASHINGTON, DC, 20036-2680, US

(73) Assignee: Rohm Co., Ltd., Kyoto-shi, Kyoto (JP)

(21) Appl. No.: 12/063,561

(22) PCT Filed: Aug. 15, 2006
(86) PCT No.: PCT/JP2006/316030

§ 371 Date: Feb. 11, 2008

(30) Foreign Application Priority Data
Aug. 18, 2005 (JP) 2005-237201

(51) Int. Cl. A61B 5/15; A61M 37/00; A61M 5/42

(52) U.S. Cl. 600/583; 604/289; 604/112; 604/116; 604/272

(57) ABSTRACT

To provide a chip for collecting analyte from a living body. Specifically, the chip includes a thin plate-shaped main body, an analyte inlet portion, and a thin portion. Analyte is introduced into the analyte inlet portion, which is formed within the main body. The thin portion partitions the space forming the analyte inlet portion from the external space. According to the chip of the present invention, it is possible to eliminate the time for collecting analyte from a living body using a hypodermic syringe or the like, and placing the collected analyte into the chip. Also, the chip according to the present invention does not leave analyte remaining at the position from which the analyte was extracted, so after the test the position where the analyte was extracted from the living body is clean.

### CROSS-REFERENCE TO RELATED APPLICATION

This U.S. National stage application claims priority under 35 U.S.C. § 119(a) to Japanese Patent Application No. 2005-237201, filed in Japan on Aug. 18, 2005, the entire contents of which are hereby incorporated herein by reference.

### BACKGROUND OF THE INVENTION

1. Field of the Invention

The present invention relates to a biochip that carries out biochemistry tests for DNA, cells, proteins, immunity, and so on. More particularly the present invention relates to a chip for collecting analyte from a living body.

2. Background Information

Blood tests are frequently carried out, such as for periodic health examinations, tests in hospitals, tests for doping of athletes, and so on. The method of collecting blood in normal blood tests is to insert a hypodermic syringe having a hollow needle into a protruding vein on an arm to which a tourniquet has been applied, and to withdraw the piston of the syringe to take the blood sample. In this method, a tourniquet is wrapped around the arm in order to make it easier to target the vein, so the person providing the sample feels a sense of pressure in the arm. Also, only a person familiar with medical treatment can insert the needle in the vein.

However, in recent years the trend towards individuals managing their own health has been increasing. For example, it is possible for subjects to take their own blood samples and carry out tests, as typified by commercial blood test kits.

Alongside this trend towards individuals increasingly managing their own health, biotechnology has developed greatly. The biochip, with which DNA, cell, protein, immunity, and other biochemical tests are carried out, is the main symbol of this development of biotechnology.

Japanese Patent Application Laid-open No. 2004-290641 discloses a method in which a hollow needle is inserted into a vein of a varicose vein to collect a sample of venous blood from the vein, and a biochip, so that the normal method of collecting a blood sample can be carried out by the person to be tested. In this method, the impedance within the vein is measured using an electrode embedded within the needle, in order to direct the needle to penetrate the vein of the varicose vein, and the impedance is monitored. The person being tested can penetrate the vein of the varicose vein with the needle in accordance with the monitored information.

Also, U.S. Pat. No. 4,883,767 discloses a biochip for analyzing blood, and a method for carrying out tests by injecting collected blood into the biochip. The biochip according to U.S. Pat. No. 4,883,767 includes a blood inlet portion into which blood is injected, a flow path through which the injected blood passes, and reagent or the like for the test, but does not include a hollow needle. Therefore, the person being tested cuts their own capillary vessel to collect capillary blood, and injects the blood into the biochip according to U.S. Pat. No. 4,883,767. The injected blood passes through the flow path within the biochip, and reacts with the reagent for the test. The person being tested can know the state of their blood the reaction result with the reagent.

However, Japanese Patent Application Laid-open No. 2004-290641 and U.S. Pat. No. 4,883,767 as described above have the following problems. In the method according to Japanese Patent Application Laid-open No. 2004-290641, the person being tested must insert the needle of the biochip into the vein of the varicose vein by him/herself while watching the monitor. Therefore, if for example the person being tested is inexperienced, it is difficult to insert the needle into the vein. Also, in the method according to Japanese Patent Application Laid-open No. 2004-290641, the impedance of the vein is measured and displayed on the monitor, so besides a biochip with a hollow needle, several other devices are necessary, such as the monitor and a control device for biochip, and so on. Therefore, blood cannot be collected without an environment in which all these devices are provided. Therefore, carrying out tests is expensive, and at the same time the places where blood can be collected are limited.

Also, in the method according to U.S. Pat. No. 4,883,767, it is difficult to efficiently inject the extracted capillary blood into the blood inlet portion of the biochip. As a method of injecting the capillary blood, absorbing the extracted capillary blood into a dropping pipette and injecting the blood into the blood inlet portion of the biochip can be considered. If this method is used, capillary blood adheres to the internal walls of the dropping pipette, so it is not possible to use all the extracted capillary blood in the test. Also, capillary blood remains at the location where the blood was extracted.

### SUMMARY OF THE INVENTION

Therefore, it is an object of the present invention to solve the above problems by providing a chip with which it is possible to simply collect analyte, such as blood for example, from a living body which is the subject being tested.

To solve the above problems, a chip is provided for collecting analyte from a living body. Specifically, the chip includes a thin plate-shaped main body, an analyte inlet portion, and a thin portion. Analyte is introduced into the analyte inlet portion, which is formed within the main body. The thin portion partitions the space forming the analyte inlet portion from the external space.

In the following, the analyte is, for example, blood, and the subject to be tested is a living body. When blood is collected from a subject to be tested, the chip is pressed so that the thin portion of the chip directly contact the skin of the subject to be tested. Then the skin of the subject being tested is pierced with a needle via the thin portion, and bleeding is caused. At this time, the blood pressure within the veins has a higher pressure than the interior of the chip. Therefore, the total quantity of the blood released flows directly into the interior of the analyte inlet portion via the thin portion. Therefore, it is possible to save the trouble of collecting the blood from the skin using a hypodermic syringe or the like, and placing the collected blood into the chip, so it is possible to easily collect and analyze blood. Also, blood does not remain on the skin after collecting the blood. Therefore, it is possible to keep the skin clean after collecting the blood.

In the chip for collecting analyte according to a second aspect of the present invention, the thin portion includes a film. When blood is collected from a subject to be tested, the chip is pressed so that the thin portion and the film of the chip directly contact the skin of the subject to be tested. In this case, it is possible to obtain greater adhesion between the chip and the skin of the subject to be tested, than in the case where the thin portion only is formed in the chip. Therefore, it is possible to introduce the blood that has been released into the analyte inlet portion without leakage.

In the chip for collecting analyte according to a third aspect of the present invention, an aperture that exposes the analyte inlet portion is formed in the main body, and the film is formed to cover the aperture. When blood is collected from a subject to be tested, the chip is pressed so that the aperture and the film of the chip directly contact the skin of the subject to be tested. Then the skin of the subject being tested is pierced with a needle via the aperture and the film, and bleeding is caused. At this time, the blood pressure within the veins has a higher pressure than the interior of the chip. Therefore, the total quantity of the blood released flows directly into the interior of the analyte inlet portion via the aperture and the film. Therefore, it is possible to save the trouble of collecting the blood from the skin using a hypodermic syringe or the like, and placing the collected blood into the chip, so it is possible to easily collect and analyze blood. Also, blood does not remain on the skin after collecting the blood. Therefore, it is possible to keep the skin clean after collecting the blood.

In the chip for collecting analyte according to a fourth aspect of the present invention, the film is coated with tacky material or adhesive. This chip adheres to the skin of the subject being tested due to the applied tacky material or adhesive. In other words, the chip is stably fixed to the skin. Therefore, after blood has been released, the blood can be more easily collected within the analyte inlet portion via the thin portion of the chip.

In the chip for collecting analyte according to a fifth aspect of the present invention, a through hole is formed that penetrates the thin portion. When blood is collected from a subject to be tested, the chip is pressed so that the thin portion and the through hole of the chip directly contacts the skin of the subject to be tested. Then the skin of the subject being tested is pierced with a needle that penetrates the thin portion, and bleeding is caused. At this time, the blood pressure within the veins has a higher pressure than the interior of the chip, so the total quantity of the blood released flows directly into the interior of the analyte inlet portion via the thin portion and the through hole of the chip. Therefore, it is possible to eliminate the time for collecting the blood from the skin using a hypodermic syringe or the like, and placing the collected blood into the chip, so it is possible to easily collect and analyze blood. Also, blood does not remain on the skin after collecting the blood. Therefore, it is possible to keep the skin clean after collecting the blood.

In the chip for collecting analyte according to a sixth aspect of the present invention, a local anesthetic agent is applied to the outer surface of the thin portion. When this chip is applied to the skin of the subject to be tested, the local anesthetic agent applied to the thin portion contacts the skin. Therefore, when the needle pierces the skin of the subject being tested and bleeding is caused, the blood can be collected without pain being felt by the subject being tested.

In the chip for collecting analyte according to a seventh aspect of the present invention, a needle portion having a needle tip that pierces the surface of the living body is further provided on an internal wall of the analyte inlet portion in a position in opposition to the thin portion. In this chip, the needle is already provided. Therefore, when collecting analyte from a subject to be tested using this chip, it is unnecessary to separately provide a needle.

In the chip for collecting analyte according to an eighth aspect of the present invention, a penetrating hole that penetrates to the analyte inlet portion is formed in a position in opposition to the thin portion. By disposing a needle in the penetrating hole formed in the main body, when collecting analyte from a subject to be tested using this chip, it is unnecessary to separately prepare the needle. Also, it is possible to maintain the pressure within the analyte inlet portion at a pressure lower than atmospheric pressure by using a suction mechanism, such as a pump or the like. Therefore, it is possible to efficiently introduce the blood of the subject being tested into the analyte inlet portion.

According to the chip of the present invention as described above, it is possible to easily collect analyte from a living body.

### BRIEF DESCRIPTION OF THE DRAWINGS

Referring now to the attached drawings which form a part of this original disclosure:

FIG. 1 is a structural diagram of a chip according to a first embodiment of the present invention;

FIG. 2 is an explanatory diagram showing a usage of the chip according to the first embodiment of the present invention;

FIG. 3 is a test example of the chip according to the first embodiment of the present invention;

FIG. 4 is a structural diagram of a chip according to a second embodiment of the present invention;

FIG. 5 is a structural diagram of a chip according to a third embodiment of the present invention;

FIG. 6 is a structural diagram of a chip according to a fourth embodiment of the present invention; and

FIG. 7 is a structural diagram of a chip according to a fifth embodiment of the present invention.

### DETAILED DESCRIPTION OF THE PREFERRED EMBODIMENTS

### First Embodiment

### (1) Structure

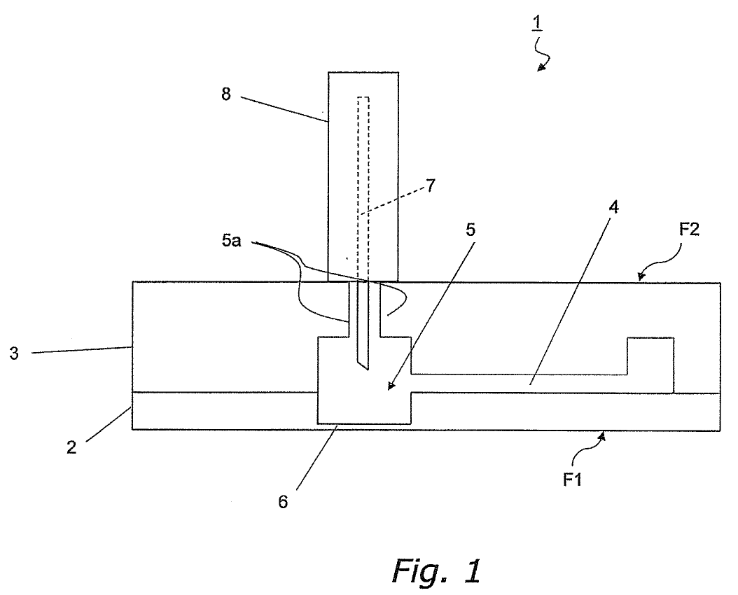

FIG. 1 is a structural diagram of a chip according to a first embodiment of the present invention. As shown in FIG. 1, a chip 1 is formed by for example bonding together two thin plate shaped substrates 2 and 3. Here, in the chip, the contact surface with the living body is called the first surface F1, and the surface opposite to the first surface F1 is called the second surface F2. The material of the substrates 2 and 3 may be for example the organic compounds indicated below, or inorganic compounds such as glass, silicon, or metal. The organic compounds can include polyethylene terephthalate (PET), polydimethylsiloxane (PDMS), polymethyl methacrylate (PMMA), polycarbonate (PC), polypropylene (PP), polystyrene (PS), poly vinyl chloride (PVC), polysiloxane, allyl ester resin, cycloolefin polymer, ST rubber, and so on.

The chip 1 has an analyte inlet portion 5, which is an internally formed space, into which analyte is introduced. The chip 1 has a thin portion 6 formed on the first surface F1 side, that partitions the space forming the analyte inlet portion 5 from the external space. In the present embodiment, a part of the substrate 2 is formed thinner than the other parts, to form the thin portion 6. The thin portion 6 has a predetermined thickness so that when the chip 1 is used, the needle 7 for piercing the living body can penetrate the thin portion 6. The needle 7 is not part of the chip 1, but is provided separately when the chip 1 is used. The predetermined thickness of the thin portion 6 depends on the material of the thin portion 6, but generally can be in the range 1 μm to 1000 μm, and in particular is preferably in the range 10 μm to 50 μm. When collecting analyte, the first surface F1 and the thin portion 6 of the chip 1 contact the skin of the living body.

Also, a penetrating hole 5a that penetrates to the analyte inlet portion 5 is formed in the second surface F2 of the chip 1. The penetrating hole 5a is for inserting the needle 7 used in collecting analyte from the living body of the subject of the test into the analyte inlet portion 5. In the present embodiment, a through hole is not provided in the thin portion 6 on the first surface F1 of the chip 1, before use. When the chip 1 is used, the needle 7 is inserted into the analyte inlet portion 5 from outside through the penetrating hole 5a, and the living body is pierced via the thin portion 6. Also, when the chip 1 is used, a needle cover 8 that covers the needle 7 is provided. The analyte obtained from the living body is introduced into the analyte inlet portion 5 through a hole opened in the thin portion 6. Further, a flow path 4 for the collected analyte is formed in the substrates 2 and 3 of the chip 1.

According to this constitution, by piercing the living body with the needle 7 via the thin portion 6, analyte from the living body is introduced into the analyte inlet portion 5 of the chip 1.

### (2) Method of Use

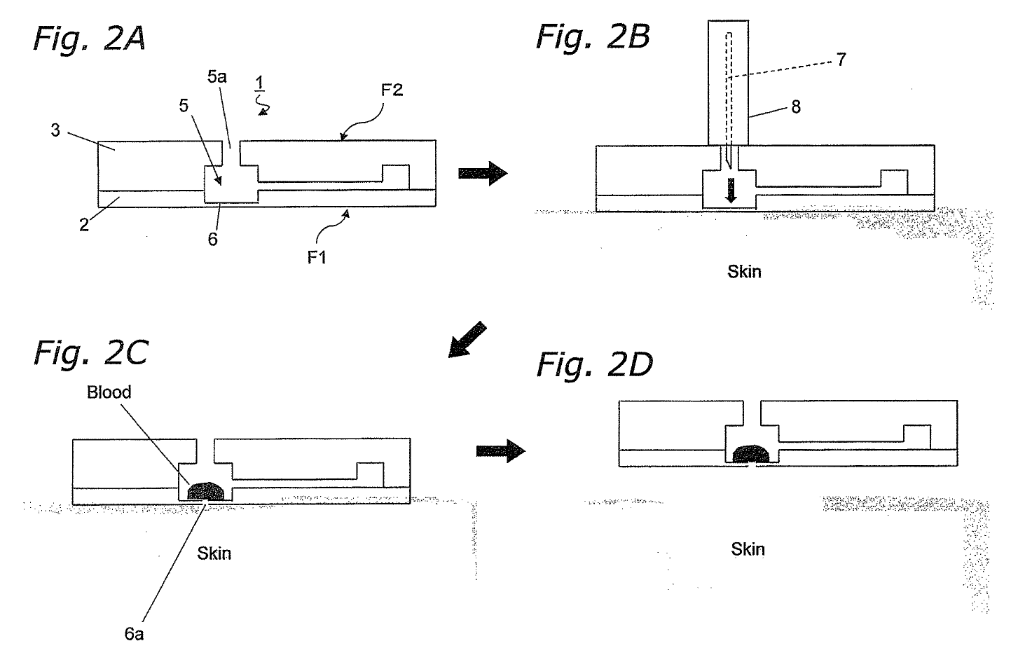

Next, the method of collecting analyte using the chip 1 according to the present embodiment is explained. In the following, in order to simplify the explanation, an example of the case where the analyte is a person's blood is taken. FIG. 2 is an explanatory diagram for the method of using the chip 1 in FIG. 1. FIG. 2A is the chip 1 before collecting blood.

FIG. 2B is the chip 1 applied closely to the surface of the skin of a person. At this time, the substrate 2 and the thin portion 6 of the chip 1 contact the surface of the skin of the person. The needle cover 8 containing the needle 7 is placed on the chip 1 so that the positions of the tip of the needle 7 within the needle cover 8 and the penetrating hole 5a are aligned. In this state, the needle 7 is pressed down by pushing the top end of the needle cover 8, and the tip of the needle 7 pierces the skin. Also, the needle 7 and the needle cover 8 are placed on the chip 1 so that when force is not applied from above, the state shown in FIG. 2B is always maintained. In other words, after the needle 7 and needle cover 8 are pressed down and the skin of the person is pierced, the needle 7 and the needle cover 8 return to the state shown in FIG. 2B. Also, piercing the living body by the needle 7 can also be done using the force of a spring housed within the needle cover 8.

FIG. 2C shows the state in which blood extracted from the surface of the skin of the person is introduced into the analyte inlet portion 5. By piercing the skin with the needle 7, a hole 6a is formed in the thin portion 6. Blood is introduced into the analyte inlet portion 5 via the hole 6a. It is because the blood pressure is higher than the pressure within the analyte inlet portion 5, and also the first surface F1 of the chip 1 is in close contact with the surface of the skin. The closer the contact between the chip 1 and the skin, the easier it is to introduce blood into the analyte inlet portion 5 without waste. After piercing by the needle 7, the needle 7 and the needle cover 8 are removed from the chip 1.

FIG. 2D shows the state in which the chip 1 is separated from the surface of the skin of the person. The hole 6a opened in the thin portion 6 is very small, so even if the chip 1 is removed from the surface of the skin, the blood introduced into the analyte inlet portion 5 does not leak from the hole 6a, but is held inside. After collecting the blood, the chip 1 containing the collected blood is placed in an analysis device such as a centrifuge, and the required analysis is carried out.

### (3) Effect

By applying the chip 1 in close contact with the surface of the living body, it is possible to directly and without waste introduce the analyte into the analyte inlet portion 5 from a hole opened in the thin portion 6. Therefore, it is possible to minimize the quantity of analyte to be collected for analysis. As a result, it is possible to easily collect analyte.

### (4) Other Constitution 1 and Effects

In the chip 1 of FIG. 1, tacky material or adhesive may be applied to the surface of the thin portion 6. This type of chip 1 has greater adhesion to the skin of the living body compared with chips 1 to which tacky material or adhesive is not applied. Therefore, after the analyte has flowed out, the analyte can be collected within the analyte inlet portion 5 through the hole 6a of the thin portion 6 with even less waste. An acrylic tacky material which has a small impact on the skin is preferable for the tacky or adhesive material, but there is no limitation on this, and any material may be used provided the adhesion between the thin membrane of the chip 1 and the skin is increased.

### (5) Other Constitution 2 and Effects

In the chip 1 of FIG. 1, a local anesthetic agent such as xylocaine may be applied to the surface of the thin portion 6. When the needle 7 pierces the skin of the living body and causes bleeding, it is possible to collect the analyte, such as blood or the like, without causing pain to be felt by the living body. There is no particular limitation on the substance used as the local anesthetic agent, provided an anesthetic effect is produced by contact with the skin.

### Example of a Test

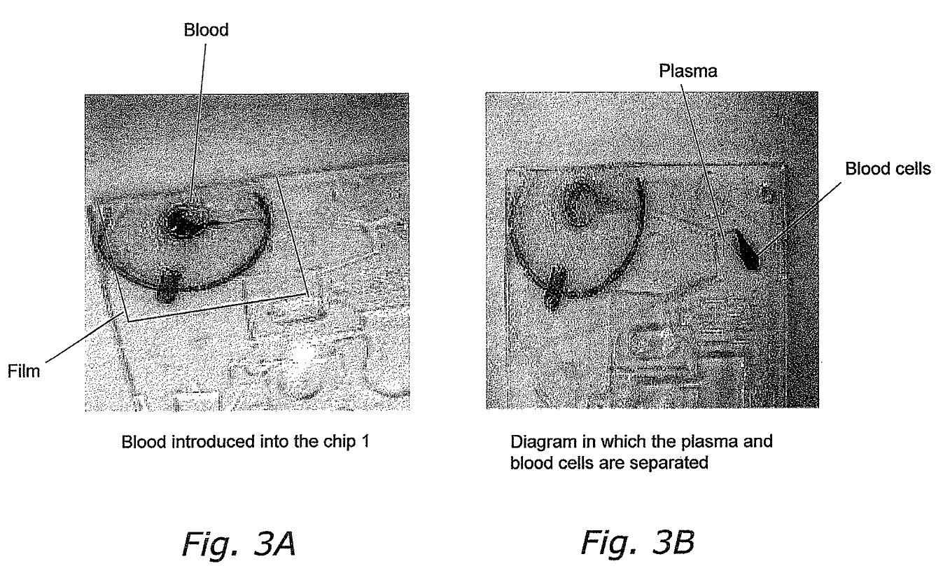

FIG. 3 is an example of a test using the chip 1 according to the first embodiment of the present invention. An acrylic tacky material was applied to the surface of the thin portion 6 that contacts the skin of the person to be tested. The chip 1 was directly pressed onto the skin of the person to be tested, and blood was collected. FIG. 3A shows the state with blood introduced into the analyte inlet portion 5. At this time, the quantity of blood collected by the chip 1 is 2.5 μl, which corresponds to the quantity required for analysis of the blood. Also, when the blood was collected and the chip was removed from the location from which the blood was taken, residual blood was not seen on the skin of the person being tested. In other words, the chip 1 was able to collect the total quantity of blood that was released from the body.

Afterwards, the chip 1 in FIG. 3A was placed in a centrifuge. FIG. 3B shows the state of the analyte after centrifugal separation. The blood within the analyte inlet portion 5 of the chip 1 was separated into plasma and blood cells within the flow path 4 formed within the substrates 2 and 3.

### Second Embodiment

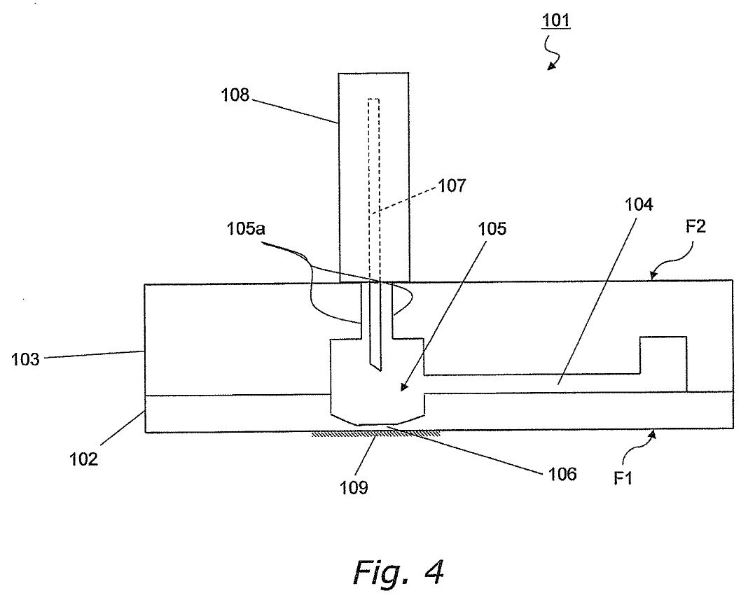

FIG. 4 is a structural diagram of a chip 101 according to a second embodiment. The chip 101 is formed by bonding together thin plate-shaped substrates 102 and 103. In the chip 101, the contact surface with the living body is called the first surface F1, and the surface opposite to the first surface F1 is called the second surface F2. The chip 101 includes an analyte inlet portion 105, and a thin portion 106. The analyte inlet portion 105 is a space formed within the substrates 102 and 103 into which analyte is introduced. The thin portion 106 is formed on the first surface F1 side of the chip 101, and partitions the space forming the analyte inlet portion 105 from the external space. Also, the thin portion 106 includes a film 109. The film 109 is formed on a part of the substrate 102 that is formed thinner than other parts. The film 109 is provided to ensure greater adhesion between the first surface F1 of the chip 1 and the skin of the living body. The film 109 is disposed in a position corresponding to the analyte inlet portion 105, and corresponding to the position that an external needle 107 penetrates when the chip 101 is used. The film 109 may have a predetermined thickness through which the needle 107 can penetrate, so that the needle 107 can penetrate the whole thin portion 106. In other words, the thin portion 106 preferably has a total thickness in the range 1 μm to 1000 μm, and preferably in the range 10 μm to 50 μm. Also, the film 109 is preferably made from a thermoplastic such as polyethylene, an elastomer such as PDMS, or a fiber such as rayon.

The film 109 may be coated or impregnated with tacky material or adhesive for greater adhesion between the skin of the living body and the chip 101. In this way the quantity of analyte leaking between the chip 101 and the living body is minimized, and the analyte can be easily introduced into the chip 101 without waste. An acrylic tacky material which has a small impact on the skin is preferable for the tacky or adhesive material, but there is no limitation on this, and any material may be used provided the adhesion with the thin membrane of the chip 101 and the skin is increased.

A local anesthetic agent such as xylocaine for example may be applied to or impregnated into the film 109. When the needle 107 pierces the skin of the living body and bleeding is caused, the analyte can be collected without pain being felt by the living body. There is no particular limitation on the substance used as the local anesthetic agent, provided an anesthetic effect is produced by contact with the skin.

A penetrating hole 105a that penetrates to the analyte inlet portion 105 is formed in the second surface F2 of the chip 101. The penetrating hole 105a is for inserting the needle 107 used in collecting analyte from the living body into the analyte inlet portion 105. When the chip 101 is used, the needle 107 is inserted into the analyte inlet portion 105 through the penetrating hole 105a from outside, and the needle 107 pierces the living body via the thin portion 106. Analyte obtained from the living body is introduced into the analyte inlet portion 105 through a hole opened in the thin portion 106.

When collecting analyte form the living body, the chip 101 is pressed so that the film 109 of the chip 101 adheres to the skin of the living body. In this case, the adhesion of the first surface F1 of the chip 101 with the skin of the living body is greater in the case where the film 109 is formed than in the case where the thin portion 106 only is formed. Therefore, it is possible to introduce the analyte released from the living body into the analyte inlet portion without leakage.

The chip 101 with this constitution has greater adhesion to the skin of the living body compared with a chip in which the thin portion only is formed. Therefore, it is possible to introduce the analyte into the analyte inlet portion 105 without leakage.

### Example of a Test

A test was carried out using the chip 101 according to the second embodiment of the present invention. A polyethylene film was used as the film 109, and an acrylic tacky material was applied to the surface of the film that contacts the skin of the person to be tested. The chip 101 was directly pressed onto the skin of the person to be tested, and 2.5 μl of blood was collected. This corresponds to the quantity necessary to analyze the blood. Also, when the blood was collected and the chip was removed from the location from which the blood was taken, residual blood was not seen on the skin of the person being tested. In other words, the chip 101 was able to collect the total quantity of blood that was released from the body.

Then the chip 101 was placed in a centrifuge. Then, the blood within the analyte inlet portion 105 of the chip 101 was separated into plasma and blood cells within the flow path 104 formed within the substrates 102 and 103.

### Third Embodiment

### (1) Constitution

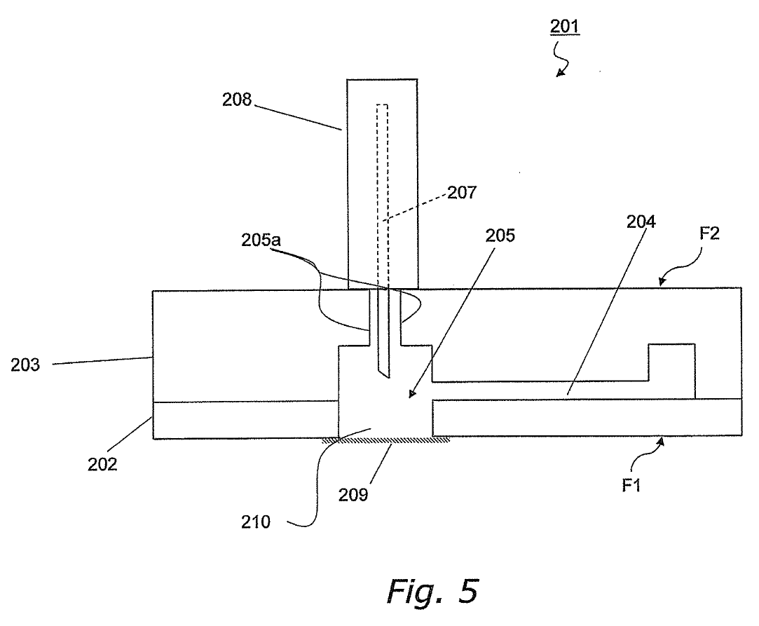

FIG. 5 is a structural diagram of a chip 201 according to a third embodiment. The chip 201 is formed by bonding together thin plate-shaped substrates 202 and 203. In the chip 201, the contact surface with the living body is called the first surface F1, and the surface opposite to the first surface F1 is called the second surface F2. The chip 201 includes an analyte inlet portion 205 and a film 209. The analyte inlet portion 205 is a space formed within the substrates 202 and 203, into which analyte is introduced. Also, an aperture 210 is formed in the first surface F1 of the chip 201 through which the analyte inlet portion 205 is exposed. The aperture 210 is formed in at least apart of the first surface F1 that contacts the living body.

The film 209 is formed to cover the aperture 210. The film 209 is provided to ensure greater adhesion between the first surface F1 of the chip 201 and skin of the living body. The thickness of the film 209 may be a thickness predetermined so that a needle 207 inserted into the chip 201 from outside can penetrate the film 209. Specifically, the thickness of the film is preferably in the range 1 μm to 1000 μm, and in particular the range 10 μm to 50 μm is preferable. Also, the film 209 is preferably made from a thermoplastic such as polyethylene, an elastomer such as PDMS, or a fiber such as rayon.

The film 209 may be coated or impregnated with tacky material or adhesive for greater adhesion between the skin of the living body and the film 209. In this way the quantity of analyte leaking between the chip 201 and the living body is minimized, and the analyte can be easily introduced into the chip 201 without waste. An acrylic tacky material which has a small impact on the skin is preferable for the tacky material, but there is no limitation on this, and any material may be used provided the adhesion with the film 209 of the chip 201 and the skin is increased.

Also, a local anesthetic agent such as xylocaine for example may be applied to or impregnated into the film 209. When the needle 207 pierces the skin of the living body and analyte is caused to flow out, the analyte can be collected without pain being felt by the living body. There is no particular limitation on the substance used as the local anesthetic agent, provided an anesthetic effect is produced by contact with the skin.

A penetrating hole 205a that penetrates to the analyte inlet portion 205 is formed in the second surface F2 of the chip 1. The penetrating hole 205a is for inserting the needle 207 used in collecting analyte from the living body into the analyte inlet portion 205. When the chip 201 is used, the needle 207 is inserted into the analyte inlet portion 205 through the penetrating hole 205a from outside, and the needle 207 pierces the living body via the thin portion 206. When the chip 201 is used, a needle cover 208 that covers the needle 207 is further provided. Analyte obtained from the living body is introduced into the analyte inlet portion 205 through a hole opened in the thin portion 206. Also, a flow path 204 for the collected analyte may be formed in the substrates 202 and 203 of the chip 201.

The method of collecting and analyzing analyte using the chip 201 having the constitution as described above is the same as the method for the first embodiment, so the explanation is omitted.

### (2) Effect

According to the constitution of this chip 201, by ensuring good adhesion of the chip 201 to the surface of the living body, it is possible to directly and without waste introduce the analyte into the analyte inlet portion 205 from the hole opened up in the film 209 and the aperture 210. Therefore, it is possible to minimize the quantity of analyte to be collected for analysis. As a result, it is possible to easily collect analyte.

### Example of a Test

A test was carried out using the chip 201 according to the third embodiment of the present invention. A polyethylene film was used as the film 209, and an acrylic tacky material was applied to the surface of the film that contacts the skin of the person to be tested. The chip 201 was directly pressed onto the skin of the person to be tested, and 2.5 μl of blood was collected. This corresponds to the quantity necessary to analyze the blood. Also, when the blood was collected and the chip was removed from the location from which the blood was taken, residual blood was not seen on the skin of the person being tested. In other words, the chip 201 was able to collect the total quantity of blood that was released from the body.

Then the chip 201 was placed in a centrifuge. Then, the blood within the analyte inlet portion 205 of the chip 201 was separated into plasma and blood cells within the flow path 204 formed within the substrates 202 and 203.

### Fourth Embodiment

### (1) Constitution

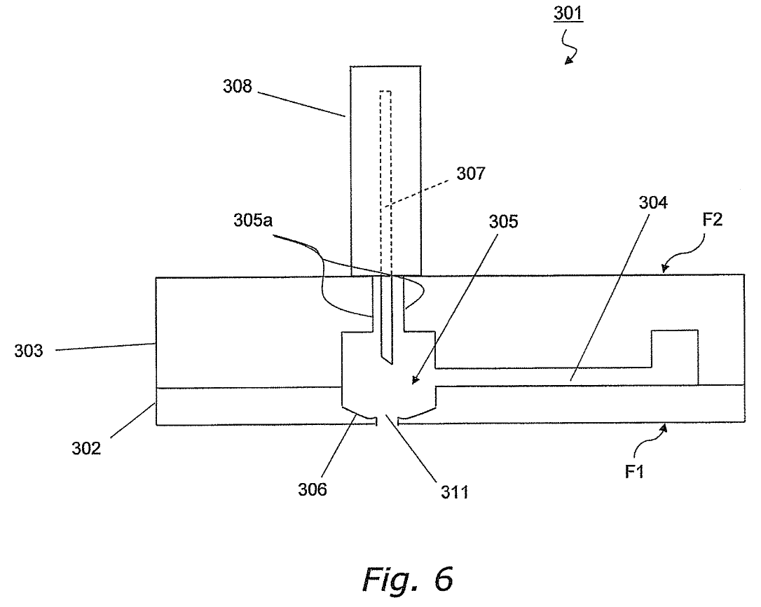

FIG. 6 is a structural diagram of a chip 301 according to a fourth embodiment. The chip 301 is formed by bonding together thin plate-shaped substrates 302 and 303. In the chip 301, the contact surface with the living body is called the first surface F1, and the surface opposite to the first surface F1 is called the second surface F2. The chip 301 includes an analyte inlet portion 305, and a thin portion 306. The analyte inlet portion 305 is a space formed within the substrates 302 and 303, into which analyte is introduced. The thin portion 306 is formed on the first surface F1 side of the chip 301, and partitions the space forming the analyte inlet portion 305 from the external space. Specifically the thin portion 306 is formed by a part of the substrate 302 that is formed thinner than other parts.

The thin portion 306 may be coated or impregnated with tacky material or adhesive for greater adhesion between the skin of the living body and the chip 301. In this way the quantity of analyte leaking between the chip 301 and the living body is minimized, and the analyte can be easily introduced into the chip 301 without waste. An acrylic tacky material which has a small impact on the skin is preferable for the adhesive material, but there is no limitation on this, and any material may be used provided the adhesion with the thin portion 306 of the chip 301 and the skin is increased.

Also, a local anesthetic agent such as xylocaine for example may be applied to or impregnated into the thin portion 306. When the needle 307 pierces the skin of the living body and analyte is caused to flow out, the analyte can be collected without pain being felt by the living body. There is no particular limitation on the substance used as the local anesthetic agent, provided an anesthetic effect is produced by contact with the skin.

A through hole 311 that penetrates the thin portion 306 is formed in the thin portion 306. The through hole 311 is formed corresponding to the position of the needle 307 that is inserted into the chip 301 from outside. The diameter of the through hole 311 may be of such size that the analyte introduced into the analyte inlet portion 305 does not leak. The diameter of the through hole 311 depends on the viscosity of the analyte, but generally the range 50 μm to 3 mm is preferable, and in particular the range 100 μm to 500 μm is preferable.

Also, a penetrating hole 305a that penetrates to the analyte inlet portion 305 is formed in the second surface F2 of the chip 301. The needle 307 used in collecting analyte from the living body is inserted in the analyte inlet portion 305. When the chip 301 is used, the needle 307 is inserted into the analyte inlet portion 305 through the penetrating hole 305a from outside, and the needle 307 pierces the living body via the through hole 311 of the thin portion 306. When the chip 301 is used, a needle cover 308 that covers the needle 307 is further provided. Analyte obtained from the living body is introduced into the analyte inlet portion 305 through the through hole 311 of the thin portion 306. Also, a flow path 304 for the collected analyte may be formed in the substrates 302 and 303 of the chip 301.

The method of collecting and analyzing analyte using the chip 301 having the constitution as described above is the same as the method for the first embodiment, so the explanation is omitted.

### (2) Effect

According to the chip 301 having this constitution, analyte is directly introduced into the analyte inlet portion 305 through the thin portion 306 and the through hole 311 of the chip 301. Therefore, it is possible to save the trouble of collecting the analyte from the skin using a hypodermic syringe or similar, and placing the collected analyte into the chip, so it is possible to easily collect the analyte. Also, analyte does not remain on the skin after collection of the analyte. Therefore, after collection of the analyte, the skin can be kept clean.

### Example of a Test

A test was carried out using the chip 301 according to the fourth embodiment of the present invention. An acrylic tacky material was applied to the surface of the thin portion 306 that contacts the skin of the person to be tested. The chip 301 was directly pressed onto the skin of the person to be tested, and 2.5 μl of blood was collected. This corresponds to the quantity necessary to analyze the blood. Also, when the blood was collected and the chip was removed from the location from which the blood was taken, residual blood was not seen on the skin of the person being tested. In other words, it was possible to collect the total quantity of blood released from the living body in the chip 301.

Then the chip 301 was placed in a centrifuge. Then, the blood within the analyte inlet portion 305 of the chip 301 was separated into plasma and blood cells within the flow path 304 formed within the substrates 302 and 303.

### Fifth Embodiment

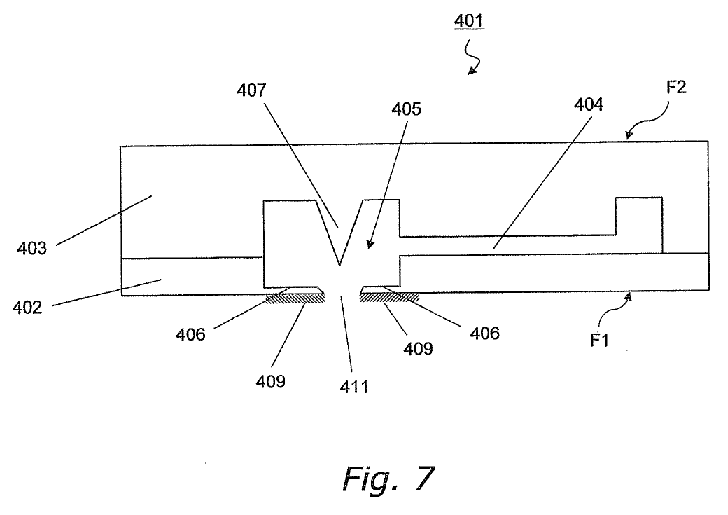

FIG. 7 is a structural diagram of a chip 401 according to a fifth embodiment of the present invention. The chip 401 is formed by bonding together thin plate-shaped substrates 402 and 403. In the chip 401, the contact surface with the living body is called the first surface F1, and the surface opposite to the first surface F1 is called the second surface F2. The chip 401 includes an analyte inlet portion 405, a thin portion 406, and a needle 407. The analyte inlet portion 405 is a space formed within the substrates 402 and 403, into which analyte is introduced. The thin portion 406 is formed on the first surface F1 side of the chip 401, and partitions the space forming the analyte inlet portion 405 from the external space. Also, the thin portion 406 includes a film 409. Also, a through hole 411 is formed in the thin portion 406 through which the analyte inlet portion 405 is exposed. The film 409 is provided on at least a part of the thin portion 406. Specifically, the film 409 is formed on a part of the substrate 402 that is formed thinner than other parts. The film 409 is provided to ensure greater adhesion between the first surface F1 of the chip 401 and the skin of the living body.

The film 409 may be coated or impregnated with tacky material or adhesive for greater adhesion between the skin of the living body and the chip 401. In this way the quantity of analyte leaking between the chip 401 and the living body is minimized, and the analyte can be easily introduced into the chip 401 without waste. An acrylic tacky material which has a small impact on the skin is preferable for the adhesive material, but there is no limitation on this, and any material may be used provided the adhesion with the thin membrane of the chip 401 and the skin is increased.

Also, a local anesthetic agent such as xylocaine for example may be applied to or impregnated into the film 409. When the needle 407 pierces the skin of the living body and release of analyte is caused, the analyte can be collected without pain being felt by the living body. There is no particular limitation on the substance used as the local anesthetic agent, provided an anesthetic effect is produced by contact with the skin. Also, it is not necessarily essential to provide the film 409 on the thin portion 406.

The needle 407 is built in to the chip 401 according to the present embodiment. The needle 407 is provided on an internal wall of the analyte inlet portion 405, at a position in opposition to the through hole 411 of the thin portion 406. Also, a flow path 404 for the collected analyte may be formed in the substrates 402 and 403 of the chip 401.

When analyte is collected using the chip 401, the chip is placed so that the film 409 of the chip 401 directly contacts the skin of the living body. Then the chip 401 is pressed from above the needle 407. Then the built in needle 407 pierces the skin through the through hole 411. In this way, analyte is introduced into the analyte inlet portion 405. According to the chip 401 having this constitution, when analyte is collected from the living body, the needle does not have to be separately provided. Therefore, it is possible for analyte to be collected by the person to be tested just by placing the chip 401 on the skin and pressing.

In the chips described under embodiments 1 to 4 above, instead of the penetrating hole that penetrates to the analyte inlet portion from outside, it is possible to apply the structure of the needle as described in the present embodiment.

### Example of a Test

A test was carried out using the chip 401 according to the fifth embodiment of the present invention. A polyethylene film was used as the film 409, and an acrylic tacky material was applied to the surface of the film that contacts the skin of the person to be tested. The chip 401 was directly pressed onto the skin of the person to be tested, and 2.5 μl of blood was collected. This corresponds to the quantity necessary to analyze the blood. Also, when the blood was collected and the chip was removed from the location from which the blood was taken, residual blood was not seen on the skin of the person being tested. In other words, the chip 401 was able to collect the total quantity of blood that was released from the body.

Then the chip 401 was placed in a centrifuge. Then, the blood within the analyte inlet portion 405 of the chip 401 was separated into plasma and blood cells within the flow path 404 formed within the substrates 402 and 403.

### INDUSTRIAL APPLICABILITY

Using the invention, it is possible to obtain a chip, in other words a biochip, that is capable of easily collecting analyte.

What is claimed is:

1. A chip for collecting analyte from a living body, comprising:
a thin plate-shaped main body;
an analyte inlet portion formed within the main body, into which analyte is introduced; and
a thin portion that partitions the space forming the analyte inlet portion from the external space.

2. The chip according to claim 1, wherein
the thin portion includes a film.

3. The chip according to claim 2, wherein
an aperture that exposes the analyte inlet portion is formed in the main body, and the film is formed to cover the aperture.

4. The chip according to claim 2, wherein
the film is coated with tacky material or adhesive.

5. The chip according to claim 1, wherein
a through hole is formed that penetrates the thin portion.

6. The chip according to claim 1, wherein
a local anesthetic agent is applied to the outer surface of the thin portion.

7. The chip according to claim 1, further comprising
a needle portion having a needle tip that pierces the surface of the living body, provided on an internal wall of the analyte inlet portion in a position in opposition to the thin portion.

8. The chip according to claim 1, wherein
a penetrating hole that penetrates to the analyte inlet portion is formed in a position in opposition to the thin portion.
